(19) United States
(12) Patent Application Publication
(10) Pub. No.: US 2025/0006338 A1
(43) Pub. Date: Jan. 2, 2025

(54) TREATMENT PLANNING FOR DELIVERING TUMOR TREATING FIELDS USING PATIENT DATA

(71) Applicant: Novocure GmbH, Root D4 (CH)

(72) Inventors: Reuven Ruby Shamir, Haifa (IL); Smadar Arvatz, Haifa (IL); Oren Peles Zeevi, Haifa (IL); Nadav Shapira, Haifa (IL)

(73) Assignee: Novocure GmbH, Root D4 (CH)

(21) Appl. No.: 18/746,363

(22) Filed: Jun. 18, 2024

Related U.S. Application Data
(60) Provisional application No. 63/524,534, filed on Jun. 30, 2023.

(51) Int. Cl. G16H 20/30; A61N 1/40

(52) U.S. Cl. CPC G16H 20/30; A61N 1/40

(57) ABSTRACT

A method for treatment planning for delivering tumor treating fields to a subject, the method comprising: obtaining a plurality of treatment locations for delivering tumor treating fields to a subject, each treatment location comprising a plurality of transducer locations for delivering tumor treating fields to the subject; for each treatment location, locating a plurality of transducers on the subject, wherein each transducer is located on the subject according to a respective transducer location for the treatment location; delivering tumor treating fields to the subject using the transducers located on the subject; recording data from delivering tumor treating fields to the subject; and removing the plurality of transducers from the subject; and selecting at least one of the treatment locations for further delivering tumor treating fields to the subject.

### CROSS-REFERENCE TO RELATED APPLICATIONS

This application claims priority to U.S. Provisional Application No. 63/524,534, filed Jun. 30, 2023, which is incorporated herein by reference in its entirety.

### BACKGROUND

Tumor treating fields (TTFields) are low intensity alternating electric fields within the intermediate frequency range (for example, 50 kHz to 1 MHZ), which may be used to treat tumors as described in U.S. Pat. No. 7,565,205. TTFields are induced non-invasively into the region of interest by transducers placed on the patient's body and applying AC voltages between the transducers. Conventionally, transducers used to generate TTFields include a plurality of electrode elements comprising ceramic disks. One side of each ceramic disk is positioned against the patient's skin, and the other side of each disc has a conductive backing. Electrical signals are applied to this conductive backing, and these signals are capacitively coupled into the patient's body through the ceramic discs. Conventional transducer designs include arrays of ceramic disks attached to a subject's body via a conductive skin-contact layer such as a hydrogel. AC voltage is applied between a pair of transducers for an interval of time to generate an electric field with field lines generally running in the front-back direction. Then, AC voltage is applied at the same frequency between at least another pair of transducers for another interval of time to generate an electric field with field lines generally running in the right-left direction. The system then repeats this two-step sequence throughout the treatment.

### BRIEF DESCRIPTION OF THE DRAWINGS

FIG. 1 depicts an example method for treatment planning for delivering TTFields to a subject.

FIG. 2 depicts an example system for delivering TTFields to a subject's body.

FIGS. 3A and 3B depict schematic views of exemplary designs of a transducer for applying alternating electric fields.

FIG. 4 depicts an example placement of transducers on a subject's head.

FIG. 5 an example computer apparatus.

### DESCRIPTION OF EMBODIMENTS

This application describes exemplary techniques for treatment planning for delivering TTFields to a subject by assessing different possible treatment locations for delivering TTFields to the subject.

Determining treatment locations for delivering TTFields to a subject can be a very complex process involving large amounts of data and intensive computational requirements. The computational solving of complex algorithms required for determining the treatment locations can take a significant amount of time. For example, determining treatment locations for delivering TTFields to the subject may require obtaining multiple medical images of a subject, processing the medical images to determine the location of a tumor in the subject, assigning conductivities to tissue types of the subject, generating a three-dimensional model of the subject, simulating applying TTFields to the subject for numerous transducer locations on the subject, calculating TTFields dosages for each of the simulated transducer locations, and selecting one or more transducer locations to deliver TTFields to the subject.

The inventors recognized that a need exists for a process that is less expensive, less computationally intensive, and/or less time consuming for treatment planning for delivering TTFields to a subject.

The inventive techniques are particularly integrated into a practical application. Exemplary treatment planning for delivering TTFields is determined based on data received for different possible treatment locations of transducers by actually delivering TTFields to the different possible treatment locations. The different possible treatment locations to be used for testing can be obtained based on information obtained regarding the subject. Data is received from using different treatment locations of transducers on the subject to determine which treatment location(s) would be ideal for the subject. For each treatment location, TTFields are delivered until at least steady state is reached for the TTFields. The data is received from sensors and the subject regarding each treatment location. The data is processed and presented for selecting one or more treatment location(s) for further application of TTFields to the subject. With the inventive techniques described herein, more transducer locations may be evaluated much quicker than with conventional techniques.

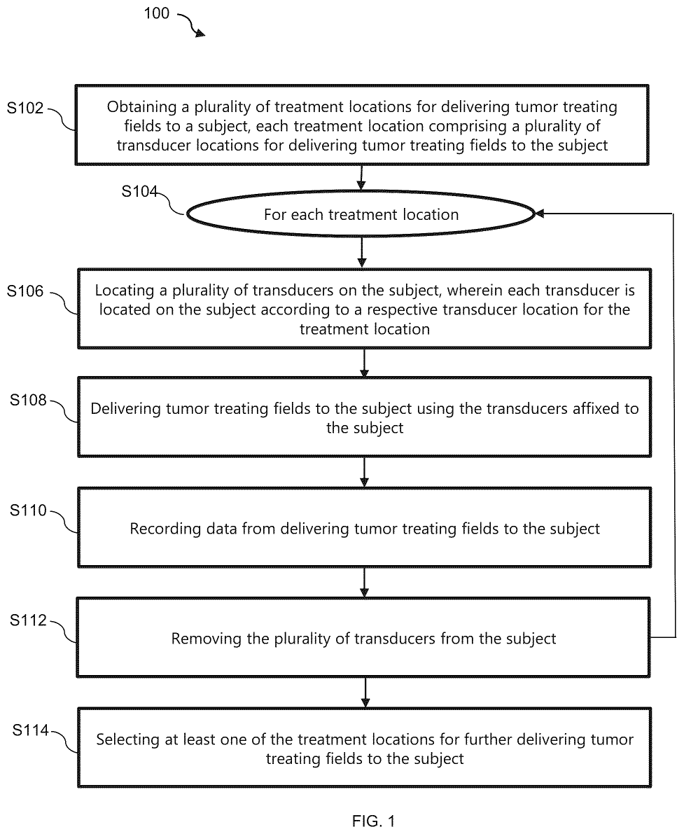

FIG. 1 depicts an example method 100 for selecting one or more treatment locations for delivering TTFields to a subject. Aspects of the method 100 may be implemented by a computer, the computer including one or more processors and memory accessible by the one or more processors, the memory storing instructions that when executed by the one or more processors cause the computer to perform certain steps of the method 100. For example, steps S102, S108, S110, and S114 may be computer implemented. Modifications, additions, or omissions may be made to method 100. While an order of operations is indicated in FIG. 1 for illustrative purposes, the timing and ordering of such operations may vary where appropriate without negating the purpose and advantages of the examples set forth in detail throughout the remainder of this disclosure.

The method 100 includes, at step S102, obtaining a plurality of treatment locations for delivering TTFields to a subject, wherein each of the plurality of treatment locations comprises a plurality of transducer locations for delivering the TTFields to the subject. For example, each of the plurality of treatment locations may include four transducer locations for delivering tumor treating fields to the subject, and each of the plurality of transducer locations may include a first pair of transducer locations and a second pair of transducer locations for alternately applying alternating electric fields to the subject. The treatment locations may be on a particular area of a subject (e.g., head, torso, abdomen, etc.) to direct the TTFields towards a region(s) of interest (such as a tumor) within the subject.

In some embodiments, the specific locations for the transducers of each treatment location obtained in step S102 may differ by at least one location for a transducer. For example, if a first treatment location has four locations 1A, 1B, 1C, and 1D to place transducers on the subject, no other treatment location can have the same four locations. As such, other treatment locations may have locations that are the same as none, one, two, or three locations of the first treatment location. As a further example, FIG. 4 may be considered as illustrating a treatment location having four locations on a head of a subject for placing four transducers 400A, 400B, 400C, and 400D. No other treatment location may have the same four locations as the transducers 400A, 400B, 400C, and 400D. Other treatment locations may have locations that are the same as none, one, two, or three locations as the transducers 400A, 400B, 400C, and 400D.

In some embodiments, the plurality of treatment locations may be based on at least one medical image of the subject (e.g., X-ray images, magnetic resonance imaging (MRI), computerized tomography (CT) images, ultrasound images, or any image providing an internal view of the subject's body). For example, the plurality of treatment locations may be generated by a computer-implemented method based on at least one medical image of the subject. In another example, the plurality of treatment locations may be selected from a database of treatment locations based on attributes of the subject. In particular, the attributes of the subject may comprise one or more of gender, race, age, height, weight, medical history, daily activity, or job type.

At step S104, the method 100 may include performing steps S106-S112, which are further described below, for each of the plurality of treatment locations obtained in step S102.

At step S106, the method 100 may include locating a plurality of transducers on the subject, wherein each of the plurality of transducers is located on the subject according to a respective transducer location for the treatment location obtained at step S102. Specifically, for each treatment location as the method loops through step S104 to step S106, the plurality of transducers may be located on the subject at the specified locations for the particular treatment location. For example, four transducers may be located on a head of the subject as illustrated in FIG. 4. For the next loop through the method, at step S106, the plurality of transducers may be located on the subject at the specified locations for the next treatment location. As discussed above, the treatment locations differ by at least one location for a transducer. In some embodiments, the transducers may be located on the subject by affixing the transducers to the subject or by placing the transducers on or next to the subject. In some embodiments, the transducers may be placed next to the subject and not affixed to the subject. For example, the transducers may be located on the subject by affixing the transducers on the subject using adhesive on a side of the transducer facing the subject. For example, the transducers may be located on the subject by placing the transducers on or next the subject using a garment worn and/or attached to the subject, where the transducers are integrated into and/or attached to the garment.

At step S108, the method 100 may include delivering the TTFields to the subject using the transducers located on the subject. In some embodiments, the tumor treating fields may be delivered for a predetermined time period, and after the predetermined time period, the tumor treating fields may be ceased for the current treatment location. The same time period may be used for each treatment location, as the method loops back from step S112 to step S104. For example, the tumor treating fields may be delivered for a sufficient time period for the subject to receive a full dosage of tumor treating fields. The time period for the subject to receive a full dosage of tumor treating fields may be based on the size, shape, and/or physical properties of subject. Due to different sizes, shapes, and physical properties of subjects, the time period for a subject to receive a full dosage of tumor treating fields may differ between subjects. In some embodiments, the time period may be the same for all subjects. For example, for each treatment location, the TTFields may be delivered for approximately 30 minutes to approximately 6 hours. As other examples, the TTFields may be delivered for each treatment location for at least approximately 30, 45, 60, 75, 90, 105, or 120 minutes.

In some embodiments, the tumor treating fields may be delivered not based on a predetermined time and instead may be based on a function of the tumor treating fields being delivered to the subject. As such, as the method loops back from step S112 to step S104 for the subject, each treatment location may have the tumor treating fields delivered for a different amount of time. As an example, for each treatment location, the tumor treating fields may be delivered based on a steady state being achieved for a measured parameter associated with the tumor treating fields. As an example, for each treatment location, the tumor treating fields may be delivered until a predetermined time period has elapsed after a steady state has been reached for delivering the tumor treating fields. As an example, for each treatment location, the tumor treating fields may be delivered until a steady state for delivering the tumor treating fields has been sustained for a predetermined time period. As an example, for each treatment location, the tumor treating fields may be delivered until at least one measured electrical parameter related to delivering tumor treating fields to the subject reaches a steady state for a predetermined time period, wherein the at least one measured electrical parameter is current or voltage. In some embodiments, the steady state for the measured electrical parameter is reached when the measured electrical parameter is constant without substantial ringing for the predetermined time period.

For example, in some embodiments, the steady state for the measured electrical parameter is reached when the measured electrical parameter is within approximately 0.2% to approximately 2%, or within approximately 0.5% to approximately 1%, or within approximately 0.2% to approximately 0.5%, or within approximately 0.3% to approximately 0.7%, of a constant value for the predetermined time period. In some embodiments, the steady state for the measured electrical parameter is reached when a measured current is within approximately 0.2% to approximately 2%, or within approximately 0.5% to approximately 1%, of a constant value for the predetermined time period. In some embodiments, the steady state for the measured electrical parameter is reached when a measured current is within approximately 0.02 amps to approximately 0.2 amps, or within approximately 0.05 amps to approximately 0.1 amps, of a constant value for the predetermined time period. In some embodiments, the steady state for the measured electrical parameter is reached when a measured voltage is within approximately 0.2% to approximately 2%, or within approximately 0.5% to approximately 1%, of a constant value for the predetermined time period. In some embodiments, the steady state for the measured electrical parameter is reached when a measured voltage is within approximately 0.02 volts to approximately 0.2 volts, or within approximately 0.05 volts to approximately 0.1 volts, of a constant value for the predetermined time period.

As an example, the predetermined time period for the steady state of the measured electrical parameter may be between approximately 1 minute and approximately 15 minutes. As an example, the predetermined time period may be between approximately 3 minutes and approximately 7 minutes.

At step S110, the method 100 may include recording data from delivering the TTFields to the subject. In some embodiments, the data may be automatically recorded in a log file while the TTFields are delivered to the subject. In one example, for each treatment location, the data recorded from delivering the TTFields to the subject may include one or more of measured voltages, measured currents, and measured temperatures from delivering the TTFields to the subject, data on the adhesion of a transducer to a subject, and subjective data, such as subject comfort information.

In some embodiments, subjective data may be obtained from the subject regarding delivering tumor treating fields to the subject. The subjective data may be obtained before, during, and/or after delivering the TTFeilds to the subject for each treatment location. For example, the subjective data may include at least one of the ability of the subject or a caregiver to affix the transducers to the subject, comfort of the subject while wearing the transducers, ability of the subject to move while the transducers are affixed to the subject, or ability of the subject or a caregiver to remove the transducers from the subject. For example, the subjective data may include states of the transducers after the transducers are removed from the subject. For example, the recorded states of the transducers after the transducers are removed from the subject may include: each transducer is in the same condition as when the transducer was affixed to the subject; at least one transducer is in a different condition as when the transducer was affixed to the subject; the number of transducers in a same condition; the number of transducers in a deteriorated condition; the number of transducers with a ripped or torn substrate; the number of transducers with an electrode element at least partially removed from the substrate; the number of transducers with missing hydrogel; the number of transducers with missing adhesive or having reduced adhesiveness. In particular, the subjective data may be recorded as a number in a range of numbers. As an example, the range of numbers for the subjective data may include a first end point and a second end point, where the first end point of the range of numbers may correspond to a most comfortable condition for the subject, and where the second end point of the range of numbers may correspond to a least comfortable condition for the subject.

At step S112, the method 100 may include removing the plurality of transducers from the subject. As noted above, the method 100 may further include repeating steps S106-S112 for each of the plurality of treatment locations obtained at step S102. In some embodiments, the plurality of transducers removed from the subject may be applied to a next treatment location for delivering the TTFields to the subject. In some embodiments, the transducers are not reused, and new transducers are applied to each treatment location for delivering the TTFields to the subject. When the steps S106-S110 are executed for each of the plurality of treatment locations, flow proceeds to step S114.

At step S114, the method 100 may include selecting at least one of the treatment locations as the desired location(s) for further delivering the TTFields to the subject. As an example, the at least one selected treatment locations may be selected based on the data recorded for each treatment location from step S110. More specifically, the selection may be based on processed data that are obtained by processing the data recorded for each treatment location from step S110. In some embodiments, the processed data may include at least one of measured current at steady state for delivering tumor treating fields to the subject, resistances calculated from measured currents and measured voltages, average measured temperatures, or subjective data above a predetermined threshold. In some embodiments, the processed data may be obtained by processing the data recorded for each treatment location and displayed simultaneously for each treatment location. The treatment locations may be ranked based on one or more of the processed data, either individually or in combination. For example, a treatment location having a highest steady state current may be selected. As another example, a treatment location having a highest normalized score based on the steady state current and the average measured temperature.

### Exemplary Apparatuses

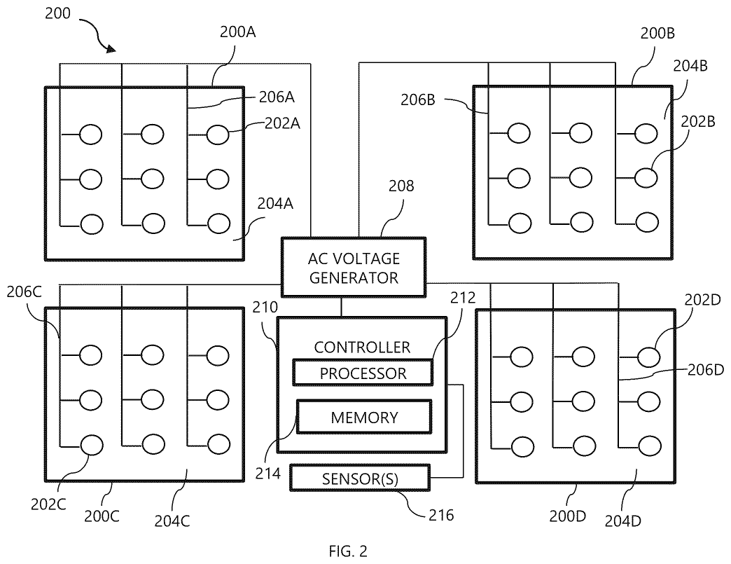

FIG. 2 depicts an example apparatus 200 to apply alternating electric fields (e.g., TTFields) to the subject's body. The system may be used for treating a target region of a subject's body with an alternating electric field. In an example, the target region may be in the subject's brain, and an alternating electric field may be delivered to the subject's body via two pairs of transducers positioned on a head of the subject's body (such as, for example, in FIG. 4, which has four transducers 400A, 400B, 400C, and 400D). In another example, the target region may be in the subject's torso, and an alternating electric field may be delivered to the subject's body via two pairs of transducers positioned on at least one of a thorax, an abdomen, or one or both thighs of the subject's body. Other transducer placements on the subject's body may be possible.

The example apparatus 200 depicts an example system having four transducers (or “transducers”) 200A-D. Each transducer 200A-D may include substantially flat electrode elements 202A-D positioned on a substrate 204A-D and electrically and physically connected (e.g., through conductive wiring 206A-D). The substrates 204A-D may include, for example, cloth, foam, flexible plastic, and/or conductive medical gel. Two transducers (e.g., 200A and 200D) may be a first pair of transducers configured to apply an alternating electric field to a target region of the subject's body. The other two transducers (e.g., 200B and 200C) may be a second pair of transducers configured to similarly apply an alternating electric field to the target region.

The transducers 200A-D may be coupled to an AC voltage generator 208, and the system may further include a controller 210 communicatively coupled to the AC voltage generator 208. The controller 210 may include a computer having one or more processors 212 and memory 214 accessible by the one or more processors. The memory 214 may store instructions that when executed by the one or more processors control the AC voltage generator 208 to induce alternating electric fields between pairs of the transducers 200A-D according to one or more voltage waveforms and/or cause the computer to perform one or more methods disclosed herein. The controller 210 may monitor operations performed by the AC voltage generator 208 (e.g., via the processor(s) 212). One or more sensor(s) 216 may be coupled to the controller 210 for providing measurement values or other information to the controller 210.

The electrode elements 202A-D may be capacitively coupled. In one example, the electrode elements 202A-D are ceramic electrode elements coupled to each other via conductive wiring 206A-D. When viewed in a direction perpendicular to its face, the ceramic electrode elements may be circular shaped or non-circular shaped. In other embodiments, the array of electrode elements are not capacitively coupled, and there is no dielectric material (such as ceramic, or high dielectric polymer layer) associated with the electrode elements.

The structure of the transducers 200A-D may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel. The transducer may be conductive or non-conductive.

The transducer may include any desired number of electrode elements (e.g., one or more electrode elements). For example, the transducer may include one, two, three, four, five, six, seven, eight, nine, ten, or more electrode elements (e.g., twenty electrode elements). Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. In certain embodiments, at least one electrode element of the first, the second, the third, or the fourth transducer may include at least one ceramic disk that is adapted to generate an alternating electric field. In non-limiting embodiments, at least one electrode element of the first, the second, the third, or the fourth transducer may include a polymer film that is adapted to generate an alternating electric field.

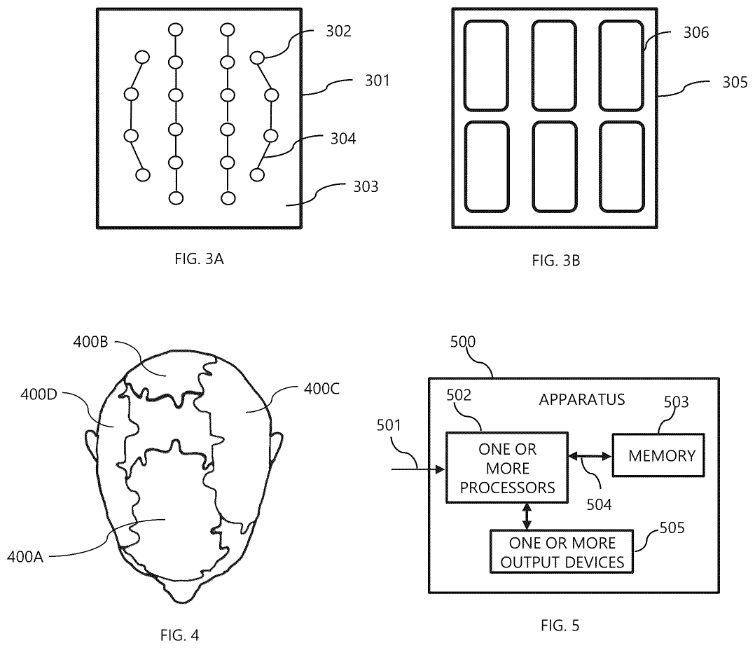

FIG. 3A illustrates a schematic view of an exemplary design of a transducer for applying alternating electric fields. The transducer 301 includes twenty electrode elements 302, which are positioned on the substrate 303, and the electrode elements 302 are electrically and mechanically connected to one another through a conductive wiring 304. In some embodiments, the electrode elements 302 can include a ceramic disk.

FIG. 3B illustrates a schematic view of an exemplary design of a transducer for applying alternating electric fields. The transducer 305 may include substantially flat electrode elements 306, respectively. In some embodiments, the electrode elements 306 are non-ceramic dielectric materials positioned over a plurality of flat conductors. Examples of non-ceramic dielectric materials positioned over flat conductors may include polymer films disposed over pads on a printed circuit board or over flat pieces of metal. In some embodiments, such polymer films have a high dielectric constant, such as, for example, a dielectric constant greater than 10. In non-limiting embodiments, the electrode elements 306 may have various shapes. For example, the electrode elements may be triangular, rectangular, circular, oval, ovaloid, ovoid, or elliptical in shape or substantially triangular, substantially rectangular, substantially circular, substantially oval, substantially ovaloid, substantially ovoid, or substantially elliptical in shape. In some embodiments, each of electrode elements 306 may have a same shape, similar shapes, and/or different shapes.

FIG. 5 depicts an example computer apparatus for use with the embodiments herein. As an example, the apparatus 500 may be a computer to implement certain inventive techniques disclosed herein, such as selecting at least one transducer layout for delivering the TTFields to a subject according to FIG. 1. For example, steps S102, S108, S110, and S114 of FIG. 1 may be performed by a computer, such as computer apparatus 500. As an example, the apparatus 500 may be used as the controller 210 of FIG. 2, or as a separate computer apparatus located remote from the controller 210. For example, steps S102, S108, S110, and S114 of FIG. 1 may be performed by a controller, such as controller 210. The apparatus 500 may include one or more processors 502, memory 503, one or more input devices, and one or more output devices 505.

In one example, based on input 501, the one or more processors 502 generate control signals to control the voltage generator. In one example, the input 501 is user input. In another example, the input 501 may be from another computer in communication with the controller apparatus 500. The memory 503 is accessible by the one or more processors 502 (e.g., via a link 504) so that the one or more processors 502 can read information from and write information to the memory 503. The memory 503 may store instructions that when executed by the one or more processors 502 implement one or more methods of the present disclosure. The memory 503 may be a non-transitory computer readable medium (or a non-transitory processor readable medium) containing a set of instructions thereon for treatment planning for delivering tumor treating fields to a subject, wherein when executed by a processor (such as one or more processors 502), the instructions cause the processor to perform one or more methods discussed herein. The input 501 may be received in conjunction with one or more input devices (not shown) of the apparatus 500.

The apparatus 500 may be an apparatus for selecting at least one transducer layout for treatment planning for delivering tumor treating fields to a subject, the apparatus including: one or more processors (such as one or more processors 502); and memory (such as memory 503) accessible by the one or more processors, the memory storing instructions that when executed by the one or more processors, cause the apparatus to perform one or more methods described herein.

The one or more output devices 505 may provide the status of the operation of the invention, such as transducer layout selection, voltages being generated, and other operational information. The output device(s) 505 may provide visualization data according to certain embodiments of the invention.

### Illustrative Embodiments

The invention includes other illustrative embodiments (“Embodiments”) as follows.

Embodiment 1: A computer-implemented method for treatment planning for delivering tumor treating fields to a subject, the method comprising: obtaining a plurality of treatment locations for delivering tumor treating fields to a subject, each treatment location comprising a plurality of transducer locations for delivering tumor treating fields to the subject;

for each treatment location, delivering tumor treating fields to the subject using transducers located on the subject, and recording data from delivering tumor treating fields to the subject; and selecting, based on the data recorded from delivering tumor treating fields to the subject, at least one of the treatment locations for further delivering tumor treating fields to the subject.

Embodiment 1A: The method of embodiment 1, further comprising, for each treatment location: locating a plurality of transducers on the subject, wherein each transducer is located on the subject according to a respective transducer location for the treatment location; and after delivering tumor treating fields to the subject using transducers located on the subject, removing the plurality of transducers from the subject.

Embodiment 2: The method of embodiment 1, wherein the plurality of treatment locations are generated by a computer-implemented method based on at least one medical image of the subject.

Embodiment 3: The method of embodiment 1, wherein the plurality of treatment locations are based on at least one medical image of the subject.

Embodiment 4: The method of embodiment 1, wherein the at least one medical image of the subject comprises a magnetic resonance image (MRI) medical image or a computer tomography (CT) medical image of the subject.

Embodiment 5: The method of embodiment 1, wherein the plurality of treatment locations are selected from a database of treatment locations based on attributes of the subject.

Embodiment 6: The method of embodiment 5, wherein the attributes of the subject comprise one or more of gender, race, age, height, weight, medical history, daily activity, or job type.

Embodiment 7: The method of embodiment 1, wherein each of the plurality of treatment locations comprises four transducer locations for delivering tumor treating fields to the subject, wherein each of the four transducer locations comprises a first pair of transducer locations and a second transducer locations for alternately applying electric fields to the subject.

Embodiment 8: The method of embodiment 1, wherein, for each treatment location, each transducer is affixed to a head of the subject.

Embodiment 9: The method of embodiment 1, wherein, for each treatment location, each transducer is affixed to a torso of the subject.

Embodiment 10: The method of embodiment 1, wherein, for each treatment location, the tumor treating fields are delivered for approximately 30 minutes to approximately 6 hours.

Embodiment 11: The method of embodiment 1, wherein, for each treatment location, the tumor treating fields are delivered for at least approximately 30, 45, 60, 75, 90, 105, or 120 minutes.

Embodiment 12: The method of embodiment 1, wherein, for each treatment location, the tumor treating fields are delivered until at least one measured electrical parameter related to delivering tumor treating fields to the subject reaches a steady state for a predetermined time period, wherein the at least one measured electrical parameter is current or voltage.

Embodiment 13: The method of embodiment 12, wherein the predetermined time period is between approximately 1 minute and approximately 15 minutes.

Embodiment 14: The method of embodiment 12, wherein the predetermined time period is between approximately 3 minutes and approximately 7 minutes.

Embodiment 15: The method of embodiment 12, wherein the steady state for the measured electrical parameter is reached when the measured electrical parameter is constant without substantial ringing for the predetermined time period.

Embodiment 16: The method of embodiment 12, wherein the steady state for the measured electrical parameter is reached when the measured electrical parameter is within approximately 1% of a constant value for the predetermined time period.

Embodiment 17: The method of embodiment 12, wherein the steady state for the measured electrical parameter is reached when at least one of: a measured current is within approximately 0.02 amps to approximately 0.2 amps of a constant value for the predetermined time period, or a measured voltage is within approximately 0.02 volts to approximately 0.2 volts of a constant value for the predetermined time period.

Embodiment 17A: The method of embodiment 12, wherein the steady state for the measured electrical parameter is reached when a measured current is within approximately 0.2% to approximately 2% of a constant value for the predetermined time period.

Embodiment 17B: The method of embodiment 12, wherein the steady state for the measured electrical parameter is reached when a measured current is within approximately 0.02amps to approximately 0.2 amps of a constant value for the predetermined time period.

Embodiment 18: The method of embodiment 12, wherein the steady state for the measured electrical parameter is reached when a measured voltage is within approximately 0.2% to approximately 2% of a constant value for the predetermined time period.

Embodiment 19: The method of embodiment 12, wherein the steady state for the measured electrical parameter is reached when a measured voltage is approximately 0.02 volts to approximately 0.2 volts of a constant value for the predetermined time period.

Embodiment 20: The method of embodiment 1, wherein, for each treatment location, the data recorded from delivering tumor treating fields to the subject comprises measured voltages and measured currents from delivering tumor treating fields to the subject.

Embodiment 21: The method of embodiment 1, wherein, for each treatment location, the data recorded from delivering tumor treating fields to the subject comprises measured temperatures from delivering tumor treating fields to the subject.

Embodiment 22: The method of embodiment 1, wherein, for each treatment location, the data recorded from delivering tumor treating fields to the subject comprises subjective data obtained from the subject regarding delivering tumor treating fields to the subject.

Embodiment 23: The method of embodiment 22, wherein the subjective data includes at least one of: ability of the subject or a caregiver to affix the transducers to the subject, comfort of the subject while wearing the transducers, ability of the subject to move while the transducers are affixed to the subject, or ability of the subject or a caregiver to remove the transducers from the subject.

Embodiment 24: The method of embodiment 22, wherein the subjective data is recorded as a number in a range of numbers, wherein a first end point of the range of numbers corresponds to a most comfortable condition for the subject, and wherein a second end point of the range of numbers corresponds to a least comfortable condition for the subject.

Embodiment 25: The method of embodiment 1, wherein, for each treatment location, the data recorded from delivering tumor treating fields to the subject comprises subject comfort information.

Embodiment 26: The method of embodiment 1, wherein, for each treatment location, the data recorded from delivering tumor treating fields to the subject comprises subjective data regarding states of the transducers after the transducers are removed from the subject.

Embodiment 27: The method of embodiment 1, wherein, for each treatment location, the data is automatically recorded in a log file while tumor treating fields are delivered to the subject.

Embodiment 28: The method of embodiment 1, further comprising processing the data recorded for each treatment location to obtain processed data, wherein selecting at least one of the treatment locations for further delivering tumor treating fields to the subject is based on the processed data.

Embodiment 29: The method of embodiment 28, wherein the processed data comprises at least one of: measured current at steady state for delivering tumor treating fields to the subject, resistances calculated from measured currents and measured voltages, average measured temperatures, or subjective data above a predetermined threshold.

Embodiment 30: The method of embodiment 1, further comprising: processing the data recorded for each treatment location to obtain processed data; and displaying the processed data simultaneously for each treatment location.

Embodiment 31: The method of embodiment 1, wherein the at least one treatment locations selected for further delivering tumor treating fields to the subject comprises a primary treatment location and a secondary treatment location.

Embodiment 32: A method for treatment planning for delivering tumor treating fields to a subject, the method comprising: for each of a plurality of treatment locations for delivering tumor treating fields to a subject, each treatment location comprising a plurality of transducer locations for delivering tumor treating fields to the subject; locating a plurality of transducers on the subject, wherein each transducer is located on the subject according to a respective transducer location for the treatment location; delivering tumor treating fields to the subject using the transducers; and recording data from delivering tumor treating fields to the subject; and selecting, based on the data recorded from delivering tumor treating fields to the subject, at least one of the treatment locations for further delivering tumor treating fields to the subject.

Embodiment 33: The method of Embodiment 35, wherein locating the plurality of transducers on the subject comprises affixing plurality of transducers to the subject.

Embodiment 34: The method of Embodiment 35, wherein locating the plurality of transducers on the subject comprises placing the plurality of transducers on the subject but not affixing the plurality of transducers to the subject.

Embodiment 35: An apparatus for treatment planning for delivering tumor treating fields to a subject, the method comprising: a voltage generator to generate voltages for delivering tumor treating fields to a subject via transducers located on the subject; one or more sensors to measure at least one parameter associated with delivering tumor treating fields to the subject; an input device to receive feedback from a subject regarding delivering tumor treating fields to the subject; and; a controller comprising: one or more processors; and memory accessible by the one or more processors, the memory storing instructions that when executed by the one or more processors, cause the controller to instruct the voltage generator to generate signals for tumor treating fields until current measured from delivering tumor treating fields to the subject reaches a steady state for a predetermined time period, wherein the memory stores a log file to record information from the one or more sensors and the input device.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A computer-implemented method for treatment planning for delivering tumor treating fields to a subject, the method comprising:
obtaining a plurality of treatment locations for delivering tumor treating fields to a subject, each treatment location comprising a plurality of transducer locations for delivering tumor treating fields to the subject;
for each treatment location,
delivering tumor treating fields to the subject using transducers located on the subject; and
recording data from delivering tumor treating fields to the subject; and
selecting, based on the data recorded from delivering tumor treating fields to the subject, at least one of the treatment locations for further delivering tumor treating fields to the subject.

2. The method of claim 1, wherein the plurality of treatment locations are generated by a computer-implemented method based on at least one medical image of the subject.

3. The method of claim 1, wherein the plurality of treatment locations are selected from a database of treatment locations based on attributes of the subject.

4. The method of claim 3, wherein the attributes of the subject comprise one or more of gender, race, age, height, weight, medical history, daily activity, or job type.

5. The method of claim 1, wherein, for each treatment location, the tumor treating fields are delivered for approximately 30 minutes to approximately 6 hours.

6. The method of claim 1, wherein, for each treatment location, the tumor treating fields are delivered until at least one measured electrical parameter related to delivering tumor treating fields to the subject reaches a steady state for a predetermined time period,
wherein the at least one measured electrical parameter is current or voltage.

7. The method of claim 6, wherein the predetermined time period is between approximately 1 minute and approximately 15 minutes.

8. The method of claim 6, wherein the steady state for the measured electrical parameter is reached when the measured electrical parameter is constant without substantial ringing for the predetermined time period.

9. The method of claim 6, wherein the steady state for the measured electrical parameter is reached when the measured electrical parameter is within approximately 0.2% to approximately 2% of a constant value for the predetermined time period.

10. The method of claim 6, wherein the steady state for the measured electrical parameter is reached when at least one of:
a measured current is within approximately 0.02 amps to approximately 0.2 amps of a constant value for the predetermined time period, or
a measured voltage is within approximately 0.02 volts to approximately 0.2 volts of a constant value for the predetermined time period.

11. The method of claim 1, wherein, for each treatment location, the data recorded from delivering tumor treating fields to the subject comprises measured voltages and measured currents from delivering tumor treating fields to the subject.

12. The method of claim 1, wherein, for each treatment location, the data recorded from delivering tumor treating fields to the subject comprises subjective data obtained from the subject regarding delivering tumor treating fields to the subject.

13. The method of claim 12, wherein the subjective data includes at least one of:
ability of the subject or a caregiver to affix the transducers to the subject,
comfort of the subject while wearing the transducers,
ability of the subject to move while the transducers are affixed to the subject, or
ability of the subject or a caregiver to remove the transducers from the subject.

14. The method of claim 1, wherein, for each treatment location, the data recorded from delivering tumor treating fields to the subject comprises subjective data regarding states of the transducers after the transducers are removed from the subject.

15. The method of claim 1, further comprising processing the data recorded for each treatment location to obtain processed data, wherein selecting at least one of the treatment locations for further delivering tumor treating fields to the subject is based on the processed data.

16. The method of claim 1, wherein further delivering tumor treating fields to the subject is for a time period to receive a full dosage of tumor treating fields.

17. A method for treatment planning for delivering tumor treating fields to a subject, the method comprising:
for each of a plurality of treatment locations for delivering tumor treating fields to a subject, each treatment location comprising a plurality of transducer locations for delivering tumor treating fields to the subject;
locating a plurality of transducers on the subject, wherein each transducer is located on the subject according to a respective transducer location for the treatment location;
delivering tumor treating fields to the subject using the transducers; and
recording data from delivering tumor treating fields to the subject; and
selecting, based on the data recorded from delivering tumor treating fields to the subject, at least one of the treatment locations for further delivering tumor treating fields to the subject.

18. The method of claim 17, wherein locating the plurality of transducers on the subject comprises affixing plurality of transducers to the subject.

19. The method of claim 17, wherein locating the plurality of transducers on the subject comprises placing the plurality of transducers on the subject but not affixing the plurality of transducers to the subject.

20. An apparatus for treatment planning for delivering tumor treating fields to a subject, the method comprising:
a voltage generator to generate voltages for delivering tumor treating fields to a subject via transducers located on the subject;
one or more sensors to measure at least one parameter associated with delivering tumor treating fields to the subject;
an input device to receive feedback from a subject regarding delivering tumor treating fields to the subject; and
a controller comprising: one or more processors; and memory accessible by the one or more processors, the memory storing instructions that when executed by the one or more processors, cause the controller to instruct the voltage generator to generate signals for tumor treating fields until current measured from delivering tumor treating fields to the subject reaches a steady state for a predetermined time period,
wherein the memory stores a log file to record information from the one or more sensors and the input device.
